(19) United States
(12) Patent Application Publication
(10) Pub. No.: US 2014/0073854 A1
(43) Pub. Date: Mar. 13, 2014

(54) ENDOSCOPE ATTACHMENT AND METHOD

(71) Applicant: LUCENT MEDICAL SYSTEMS, INC., Kirkland, WA (US)

(72) Inventors: Steve Vincent, Kirkland, WA (US); Curtis S. King, Kirkland, WA (US); Robert N. Golden, Kirkland, WA (US); Fred E. Silverstein, Seattle, WA (US)

(73) Assignee: LUCENT MEDICAL SYSTEMS, INC., Kirkland, WA (US)

(21) Appl. No.: 13/801,781

(22) Filed: Mar. 13, 2013

Related U.S. Application Data
(60) Provisional application No. 61/699,200, filed on Sep. 10, 2012.

(51) Int. Cl. A61B 1/018

(52) U.S. Cl. CPC A61B 1/018
USPC 600/104

(57) ABSTRACT

An endoscopic device has a tubular body with a retention channel substantially extending axially along the tube body. A medical tube is removably arranged in the retention channel and held in place by friction. A cable assembly is arranged between the medical tube and the surface of the retention channel. The cable assembly includes a shaped end fixture that is drawn between the medical tube and the retention channel when the cable is pulled, thereby releasing the medical tube from the tube body.

### CROSS-REFERENCE TO RELATED APPLICATION

This application claims the benefit under 35 U.S.C. §119(e) of U.S. Provisional Patent Application No. 61/699,200 filed Sep. 10, 2012, where this provisional application is incorporated herein by reference in its entirety.

### BACKGROUND

1. Technical Field

The present disclosure generally relates to an attachment for an endoscope type instrument and method to use the attachment. More particularly, but not exclusively, the present disclosure relates to placing a device such as a medical tube in the body of a patient using an apparatus integrated with or attached to an endoscopic-type instrument.

2. Description of the Related Art

In many medical procedures, it is often beneficial to place a medical device into the body of a patient. For example, one common medical practice is placement of a medical tube, which is carried out using an endoscope (e.g., bronchoscope, colonoscope, gastrointestinal endoscope, nasopharyngoscope, sigmoidoscope, and the like). Conventionally, a medical tube is placed by a medical practitioner who inserts the tube through the patient's mouth, nasal cavity, or by some other entry point. Typically, an endoscope includes a functional control mechanism and a camera, which provides mechanical assistance for the placement of the tube and visual confirmation to the medical practitioner that the tube has been placed at an acceptable location.

One type of medical tube procedure is a catheter placement within the human body. The procedure can be difficult. In many cases, unless the target site can be visualized directly or unless there is a pathway that is reasonably direct and confined, the medical tube placement procedure can be challenging. On the other hand, in an area of sufficient size, positioning a medical tube or another medical device under endoscopic guidance is an effective installation technique. The endoscope provides the tools for direct visualization, mechanical control (e.g., steering and bending), and access ports through which tools (e.g., air, water, suction), tubes, and other devices may be passed.

In an endoscopic guided procedure, a catheter can be installed through a tool port of the endoscope. Alternatively, the catheter can be grabbed by a snare tool and held against an endoscope surface for positioning.

Placing a catheter with an endoscopic tool channel has certain drawbacks. For example, the size of the catheter that can be placed is limited by the strict size dimensions of the endoscope's tool channel. A catheter that fits in the tool channel must have an outer diameter that is sufficiently small so as to allow adequate mechanical clearance for the endoscope to be smoothly removed while the catheter remains in place. Further, any adapter fittings of the catheter must also be sized to pass through the channel. If the endoscope and associated tool channel diameter is small, adequate space for mechanical clearance of the catheter will not be available.

In a different procedure, a catheter or other medical device can be placed using an endoscope and a snare. In this different procedure, a snare type “grabbing” or “holding” tool is placed in a tool channel of the endoscope. The catheter (or other medical device) is “grabbed” by the tool at the distal end of the endoscope and the body of the catheter is located laterally alongside the endoscope's flexible insertion tube. Subsequently, the catheter is passed into the patient at the same time the endoscope is passed into the patient. When the catheter reaches the point that the medical practitioner determines is desirable, the holding tool releases the catheter, and the endoscope is removed from the patient leaving the catheter behind.

Placing a catheter using a snare device in an endoscopic tool channel also has certain drawbacks. In this procedure, the snare type tool is passed through a working channel of the endoscope, and the snare is used to grab and hold the catheter. The snare approach reduces the size concerns that arise when a catheter is placed in an endoscopic tool channel and the endoscope is withdrawn over the catheter, but the snare approach increases the overall complexity of the procedure. For example, in many cases, the addition of the snare tool, which has actuation and control independent of the endoscope control, adds the possibility that the tube may be dropped during transit. Moreover, the medical device that rides alongside the endoscope increases the volume and surface area of foreign material passed into the patient.

### BRIEF SUMMARY

In accordance with some embodiments described herein, an endoscopic device has a tubular body with a retention channel substantially extending axially along the tubular body. A device, such as a medical tube, is removably arranged in the retention channel and held in place by friction. A cable assembly is arranged between the medical tube and the surface of the retention channel. The cable assembly includes a shaped end fixture that is drawn between the medical tube and the retention channel when the cable is pulled, thereby releasing the medical tube from the tubular body.

In another embodiment, an endoscopic device has a tubular body having a retention channel substantially extending axially along the tubular body. A medical device is removably arranged in the retention channel and held in place by friction. A separation mechanism is arranged between the medical tube and a surface of the retention channel, in some cases, the separation mechanism includes a cable assembly and a shaped end fixture. The shaped end fixture may include a ball shape, a teardrop shape, a wedge shape, or some other shape. The separation mechanism in some embodiments include a cable assembly and a temporary bond between the retention channel and the medical device.

In some embodiments the tubular body of the endoscopic device includes a plurality of retention channels. In some embodiments the tubular body is a separate component from an endoscope, and the tubular body and the endoscope are assembled together as an integrated medical device delivery tool.

In another embodiment, a method to place a medical tube in a patient's body with an endoscope is disclosed. In the method, the endoscope has arranged therewith a tubular body structure. The tubular body structure has an integrated retention channel substantially extending axially along the tubular body structure. A medical tube is removably fit in the retention channel and held in place by friction. A separation mechanism is arranged between the medical tube and a surface of the retention channel, wherein the separation mechanism includes a cable assembly. The method includes the acts of the positioning a distal end of the endoscope in a patient's body, drawing the cable assembly in a direction away from the patient such that the drawing is performed to release the medical tube from the retention channel, and removing the endoscope from the patient. In one aspect, a liquid is passed through the medical tube. In one aspect, the method includes steering a distal end of the endoscope by manipulating at least one linkage at a proximal end of the endoscope. In yet one more aspect, the method includes drawing a second cable assembly in a direction away from the patient, the drawing performed to pull a shaped end fixture between a second medical tube and a surface of a second retention channel thereby releasing the second medical tube from the second retention channel.

In another embodiment a medical device delivery tool includes a substantially tubular body having a retention channel formed therein. The retention channel is arranged to receive a medical device. A medical device is removably arranged in the retention channel and held in place by friction, and a separation mechanism is arranged between the medical device and a surface of the retention channel. In one aspect, the substantially tubular body has a secondary channel formed within the retention channel the secondary channel extends axially along the tubular body, and the secondary channel is arranged to receive a cable assembly of the separation mechanism. In some embodiments, the cable assembly includes a shaped end fixture. The shaped end fixture is arranged to be drawn between the medical device in the retention channel. In one embodiment, the cable assembly includes a stainless steel cable. In one embodiment, the substantially tubular body has a plurality of retention channels.

In another embodiment, a medical device delivery tool also includes a semi-cylindrical coupling mechanism having a selective diameter. This semi-cylindrical coupling mechanism is arranged to be removably attached to a substantially tubular body. The selective diameter of this semi-cylindrical coupling mechanism in some embodiments is between 10 and 20 mm. In an embodiment, the substantially tubular body is a body of an endoscope. In another embodiment, the substantially tubular body is a solid flexible rod.

These features with other objects and advantages which will become subsequently apparent reside in the details of construction and operation as more fully described hereafter and claimed, reference being had to the accompanying drawings forming a part hereof.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. One or more embodiments are described hereinafter with reference to the accompanying drawings in which:

FIG. 1 illustrates one embodiment of a steerable endoscope with a channel and a cooperative attachment;

FIG. 2 illustrates another embodiment of a steerable endoscope with a channel and a cooperative attachment;

FIG. 3 illustrates a method of operating the steerable endoscope with a channel and the cooperative attachment of FIG. 2;

FIGS. 4A-4D illustrate additional embodiments of steerable endoscopes with at least one channel and a cooperative attachment;

FIG. 5 illustrates a segment of an endoscope tube body having an attached preformed stiffening member;

FIG. 6 illustrates another segment of an endoscope tube body having an attached preformed stiffening member;

FIG. 7 illustrates a segment of an endoscope tube body and a cross-section A of the endoscope tube body;

FIG. 8 illustrates two additional embodiments of preformed stiffening members;

FIG. 9 is a perspective view of an endoscope tube body segment having a soft outer portion and a rigid or semi-rigid inner portion;

FIG. 10 includes top, bottom, and cross-section views of the endoscope tube body segment of FIG. 9;

FIG. 11A is a perspective view of a distal end of an endoscopic device having attached thereto a medical device delivery tool;

FIG. 11B is a side view, section view of the medical device delivery tool apparatus embodiment of FIG. 11A;

FIG. 12A is a perspective view of a distal end of a medical device placement tool arrangement;

FIG. 12B is a side view of the medical device placement tool arrangement embodiment of FIG. 12A; and

FIGS. 13A and 13B are, respectively, cross-sectional views of two different medical device delivery tool embodiments.

### DETAILED DESCRIPTION

Embodiments in the present disclosure describe devices and methods of delivering independent, external catheter tubes and other medical devices to a location in a patient's body. Several embodiments are described herein with respect to using direct endoscopic visualization, but the concepts and devices described and illustrated are not so limited. In fact, in addition or as an alternative to endoscopes (e.g., bronchoscopes, colonoscopes, gastrointestinal endoscopes, nasopharyngoscopes, sigmoidoscopes, and the like), other non-endoscopic medical device delivery tools can be used. Accordingly, an endoscope or some other medical device delivery tool can be used in cooperation with the novel attachment/detachment concepts described herein.

Once a medical device (e.g., a catheter) is put into a desirable position in the patient's body, a method to release the medical device from the medical device delivery tool is executed. The method permits the medical device delivery tool (e.g., endoscope) to be withdrawn while the medical device (e.g. catheter tube) remains in the location where it was placed.

Embodiments describe one or more medical devices such as auxiliary tubes or catheters temporarily affixed to an outer surface of a medical device delivery tool (e.g., an endoscope). Using an endoscope, an auxiliary tube can be placed under direct visualization at a specific location in the patient's body. Using non-endscopic medical device delivery tools and other visualization techniques (e.g., x-ray, fluoroscope, ultrasound, and the like), the auxiliary tube can also be placed at a desired location in a patient's body. A technique to dislocate the auxiliary tube after placement provides a simple and effective approach for the independent placement or removal of the individual components of an endoscope or other visualization system.

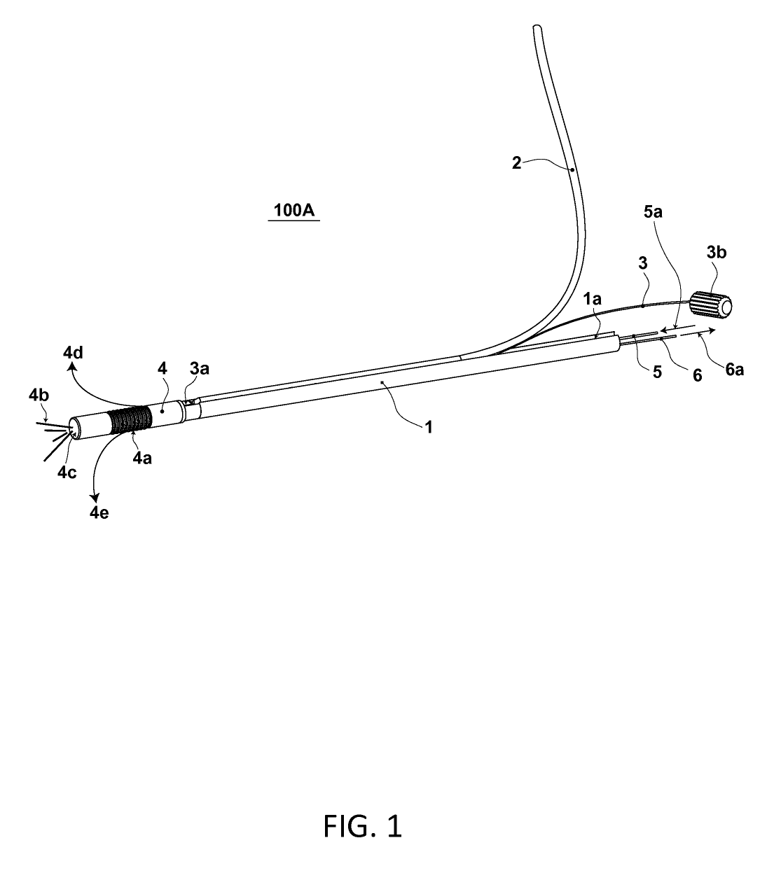

Referring now to the drawings in detail, FIG. 1 illustrates one embodiment of a steerable endoscope 100A having a channel and a cooperative attachment. A tubular body 1 of the steerable endoscope 100A has an integrated retention channel 1a formed or otherwise coupled to its outer surface. The retention channel 1a in FIG. 1 includes therein an auxiliary tube 2. A cable assembly 3 is positioned between the auxiliary tube 2 and the bottom of the retention channel 1a. A distal end of the cable assembly 3 includes a shaped end fixture 3a. A proximal end of the cable assembly 3 includes a pull handle 3b. The distal end of the steerable endoscope 100A includes a flexible end assembly 4. The flexible end assembly 4 has flexible baffles 4a to define a bending profile for the endoscope tip. A light source (not shown) can shine light 4b through a window 4c. The light permits the medical practitioner to see inside the cavity where the endoscope 100A has been placed. The flexible tip 4 can be moved in a first direction 4d or second direction 4e when a steering mechanism 5, 6 is manipulated. The steering mechanism 5, 6 may be configured to include one or more cables, cords, linkages, and any combination of these or other mechanical control devices arranged to directionally guide the flexible tip 4 of the endoscope 100A. For simplicity, the steering mechanism 5, 6 are herein referred to as a first and second steering linkage 5, 6. The first and second steering linkages 5, 6 can be manipulated by applying a pushing or pulling force 5a, 6a respectively. It is to be appreciated that the pushing and pulling mechanisms are non-limiting, and such descriptions are applied for an understanding of the embodiment illustrated in FIG. 1 and other figures. The flexible end assembly 4 can in some cases be steered with only pulling forces, with only pushing forces, with electrically actuated forces, or with any combination of mechanisms singly or in combination.

It is to be further appreciated that the cable assembly is non-limiting, and the cable assembly descriptions are applied for an understanding of the embodiment illustrated in FIG. 1 and other figures. The cable assembly is a separation mechanism configured to separate a medical tool from a retention channel. The illustrated cable assembly 3 (i.e., the separation mechanism) can be formed as a cable, wire, string, rope, cord, chain, or by some other structure positioned in the retention channel 1a below the auxiliary tube 2 and having attached thereto a shaped end fixture 3b. The cable assembly can be formed from stainless steel, polyethylene, nylon, vinyl, and many other materials. In some cases, the pull handle 3b is optional and not included.

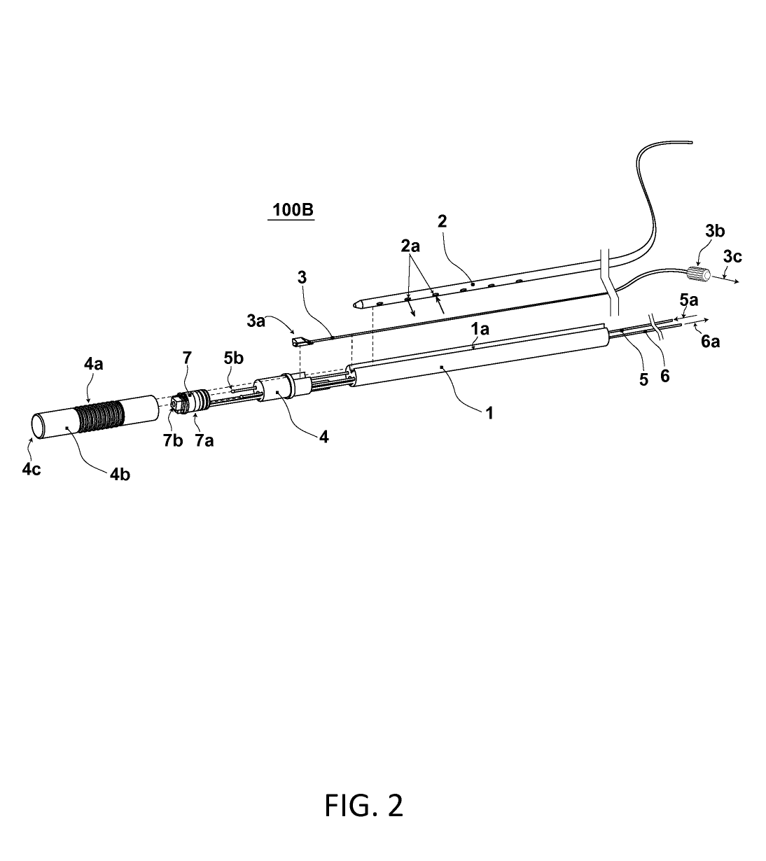

FIG. 2 illustrates another embodiment of a steerable endoscope 1008 having a channel and a cooperative attachment. Various components of the endoscope 100B are illustrated in an exploded view as represented with dashed lines. A tubular body 1 of the steerable endoscope 1008 has an integrated retention channel 1a formed or otherwise coupled to its outer surface. The retention channel 1a in FIG. 2 includes therein an auxiliary tube 2 having input or output ports 2a. A cable assembly 3 is positioned between the auxiliary tube 2 and the bottom of the retention channel 1a. A distal end of the cable assembly 3 includes a shaped end fixture 3a. A proximal end of the cable assembly 3 includes an optional pull handle 3b having a direction of pull 3c for the cable assembly 3.

The distal end of the steerable endoscope 100B includes a flexible end assembly 4, which may optionally include a rigid tip. In some embodiments, the flexible end assembly 4 has flexible baffles 4a to define a bending profile for the endoscope tip. In other embodiments, the flexible baffles 4 are a sheath covering a bending mechanism. An imaging/illumination module 7 is arranged in the rigid tip of the flexible end assembly 4. The imaging/illumination module 7 includes a light source 7b, which may also include an imaging module such as a charge couple device (CCD) video camera integrated circuit (IC). The light source may include one or more light emitting diodes (LEDs), which can shine light through the window 4c. The imaging/illumination module 7 also includes an electrical interface connector assembly 7a to provide power and control signals to the light source and optional electronic camera. The light in the endoscope 100B permits the medical practitioner to see inside the cavity where the endoscope 100B has been placed, and the camera, if included, records still pictures or a video stream of the area in front of the endoscope's flexible end assembly 4. The flexible tip 4 can be moved in a plurality of directions when steering linkages 5, 6 are manipulated and the endoscope 100B is rotated, advance, or withdrawn. The first and second steering linkages 5, 6 can be manipulated by applying pushing or pulling forces, illustrated in FIG. 2 as 5a, 6a. As seen in FIG. 2, the steering linkages 5, 6 pass through a lumens within the endoscope body 1. In some cases, the linkages 5, 6 operate independently, and in other cases, the linkages 5, 6 are coupled together or may be a single cable such that when one end of the steering linkage 5 is pulled at the proximal end of the endoscope body 1, the other end of the steering linkage 6 is pulled into the endoscope body 1.

In some embodiments, the shaped end fixture 3a has a tear drop shape. In FIG. 2, the end fixture 3a has a wedge shape. Other shapes may be formed as well. The cable assembly 3 may be formed of stainless steel, plastic, or some other material.

The auxiliary tube 2 in FIG. 2 is temporarily affixed in the retention channel 1a above the cable assembly 3 by friction or other mechanical means. Other implements may optionally be placed in the retention channel too.

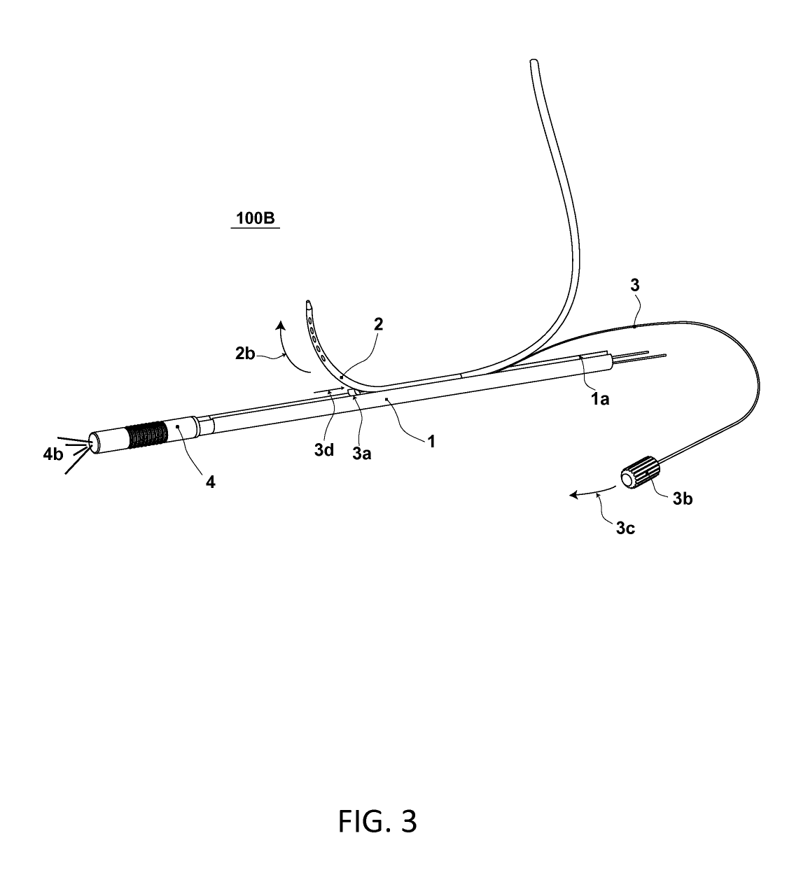

FIG. 3 illustrates a method of operating the steerable endoscope 100B having the channel 1a and cooperative attachment 2 of FIG. 2. Using the endoscope 1008 for visualization, it is possible to place the distal end of the endoscope 100B at a chosen site in the body. In the procedure, the end assembly 4 of the endoscope 100B is placed in the desired position, and the cable assembly 3 beneath the auxiliary tube 2 can be removed. The cable assembly 3 is removed by pulling the pull handle 3b in direction 3c. The shaped end fixture is thus pulled in direction 3d from the distal end of retention channel 1a toward the proximal end of the channel causing the auxiliary tube 2 to become dislodged 2b. Once the auxiliary tube 2 is free from the retention channel 1a, the endoscope 1008 can be removed independently of the auxiliary tube 2 or other implement that was arranged in the retention channel.

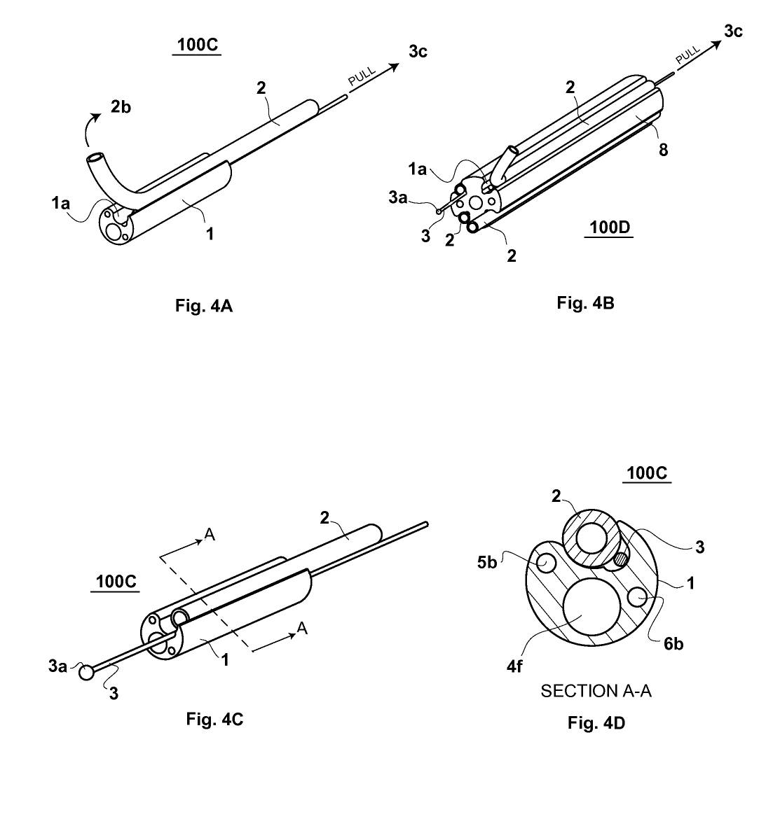

FIGS. 4A-4D illustrate additional embodiments of medical device delivery tools such as steerable endoscopes that have at least one channel and a cooperative attachment. In the embodiment of FIG. 4A, the endoscope 100C has a primary flexible core component 1. In the embodiment, a cable assembly 3 is pulled in direction 3c, and the auxiliary tube 2 is released from the retention channel 1a. In the embodiment, the medical device delivery tool of endoscope 100C is defined by the structure of the primary flexible core component 1. For the sake of simplicity, the entire length of flexible, tubular material of endoscope 100C is not shown. Instead, only the flexible core component, which is optionally formed or constructed at the distal end of endoscope 100C, is shown. Two additional aspects of the medical device delivery tool of endoscope 100C are illustrated in FIG. 4C and FIG. 4D.

The medical device delivery tool of endoscope 100C of FIG. 4C illustrates the retention cable 3 having the shaped end fixture 3a illustratively extending beyond the end of endoscope 100C. The auxiliary tube 2 is pressure fit and frictionally retained in the retention channel 1a. Cross-sectional line A-A represents where the medical device delivery tool of endoscope 100C is cut away for illustration in FIG. 4D.

FIG. 4D illustrates a cross-sectional view of the medical device delivery tool of endoscope 100C. The flexible core 1 may act as an integrated part of the main body of the endoscope 100C, or the core may be added as a tubular body of an existing endoscope. The flexible core 1 includes a primary axial through-port 4f extending the full length of the core 1 and at least one secondary port 5b (e.g., steering cable lumen) extending the same length. FIG. 4D illustrates another optional secondary port 6b. The outer surface of the core 1 has a generally smooth profile with the exception illustrated in FIG. 4D as the semi-circular cutout section view of retention channel 1a. In the embodiment, retention channel 1a extends substantially along the full length of the core 1. This retention channel 1a accommodates the press fit installation of at least one auxiliary tube 2, and the free fit installation of a single cable 3 beneath the tube.

Considering FIGS. 4A, 4C, and 4D, a medical device, such as a medical tube, can be placed in the body of a patient. After the medical practitioner uses the endoscope to determine an appropriate position for the medical tube 2, the cable 3 is pulled in direction 3c toward the distal end of the flexible core 1. The oversized end fixture 3a (e.g., the ball shaped protrusion shown in FIG. 4C) contacts the tube 2, and causes the tube 2 to dislodge from its position in the retention channel 1a. When the cable's end fixture 3a is drawn through the length of the retention channel 1a where the auxiliary tube 2 is present, the auxiliary tube 2 is released from the endoscope 100C. Subsequently, the core 1 can be removed from the patient while the medical tube 2 remains in place.

In FIG. 4B, another endoscope embodiment 100D is illustrated. The endoscope 100D includes a flexible (or rigid) tube body 8 having a plurality of medical tube retention channels 1a, each illustrated as having an optional medical tube 2 and cable assembly 3. Embodiments of other medical device delivery tools (e.g., endoscope tubes) may have more or fewer integrated retention channels.

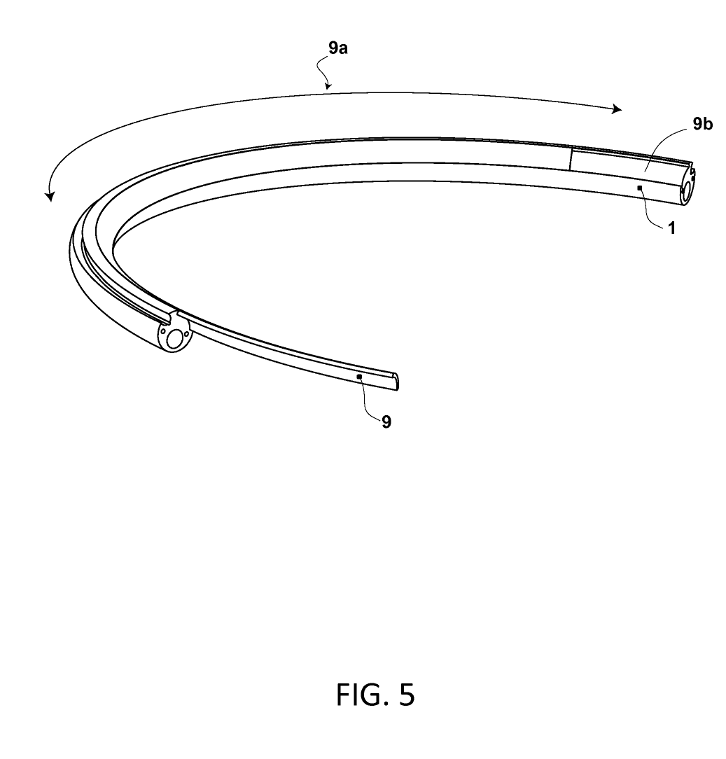

FIGS. 5-8 illustrate additional medical device delivery tool embodiments of optional structures that may be compatible with the endoscope embodiments of FIGS. 1-4. Some of the optional structures include attachments having fixed or variable stiffening properties. Other embodiments are formed as pre-shaped members that attach to the outer surface of an endoscope and conform the body of the endoscope tubular body to a desirable shape. In general, the optional attachments can be used to add stiffness to the endoscope tubular body, improve torque stability of the endoscope tubular body, create areas of regional flexibility or stiffness of the endoscope tubular body, shape the endoscope tubular body in such a way that the passage of the scope into a specific location within the patient is improved, and inclusively or alternatively perform a combination of these and other functions.

In cases where the optional attachment is used to bias the endoscope tubular body toward a pre-selected, desirable shape, the overall stiffness of the attachment and other effects (e.g., from an adhesive bond or mechanical attachment) will meet or exceed the stiffness of the endoscope tubular body within a particular range. That is, stiffness of the attachment will be arranged to account for and overcome the inherent stiffness of the attachment as including the actual or projected stiffness of the tube, the contents of the tube, stiffness caused by other attachments to the tube, ambient and/or in-use temperature, and other factors when formed in a specific configuration. For example, to simply increase the overall stiffness of the instrument, the stiffness of the optional attachment could be controlled to provide a desired mechanical performance. Furthermore, in some embodiments, the optional attachment may or may not extend the entire length of the endoscope tubular body. An optional attachment that has a lower length than the length of the endoscope tube body can be used to provide a desired regional stiffness along the endoscope tube body. In still other embodiments, the optional attachment can be formed to provide different stiffness properties over different parts of the tube body length.

FIG. 5 illustrates a segment of an endoscope tubular body 1 having an attached preformed stiffening member 9. The preformed stiffening member 9 is an optional attachment to the endoscope tubular body 1, and when attached, the preformed stiffening member arranges the flexible endoscope tubular body 1 with a particular bend or curved profile 9a. In the embodiment of FIG. 5, the preformed stiffening member 9 is mechanically arranged in a preformed stiffening member channel 9b. Other mechanisms to affix the preformed stiffening member 9 to the endoscope tubular body 1 could be used.

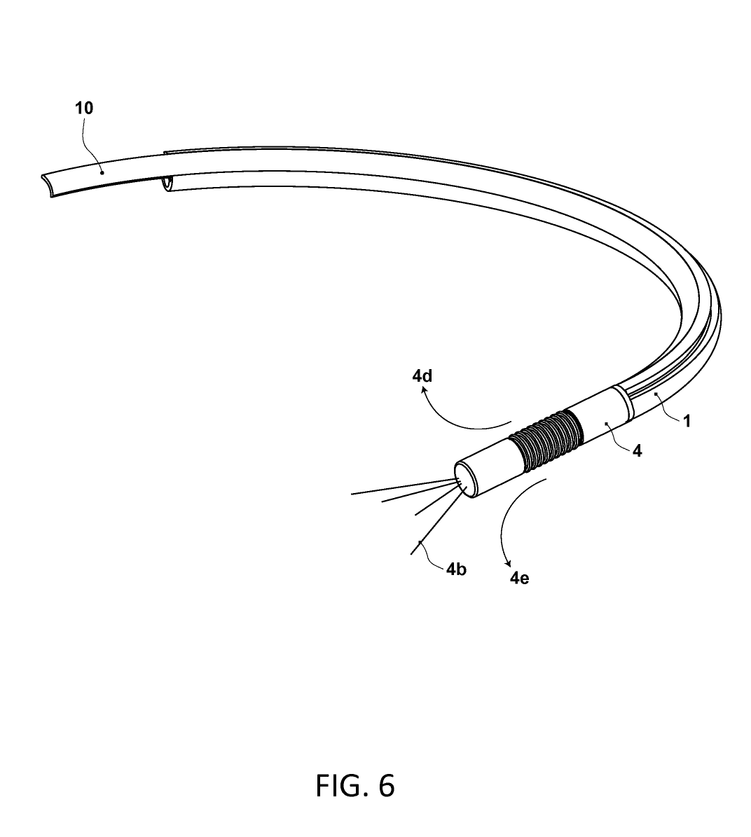

FIG. 6 illustrates another segment of an endoscope tubular body 1 having an attached preformed stiffening member 10. The endoscope tubular body 1 includes a flexible end assembly 4 that can be desirably and controllably moved in first direction 4d and a second direction 4e. When a medical practitioner selectively manipulates a steering mechanism in the proximal end of the endoscope, the flexible end assembly 4 can be moved in a selected direction within a 360 degree range of motion. The endoscope tubular body 1 of FIG. 6 includes an illumination module that can provide illumination 4b from its tip.

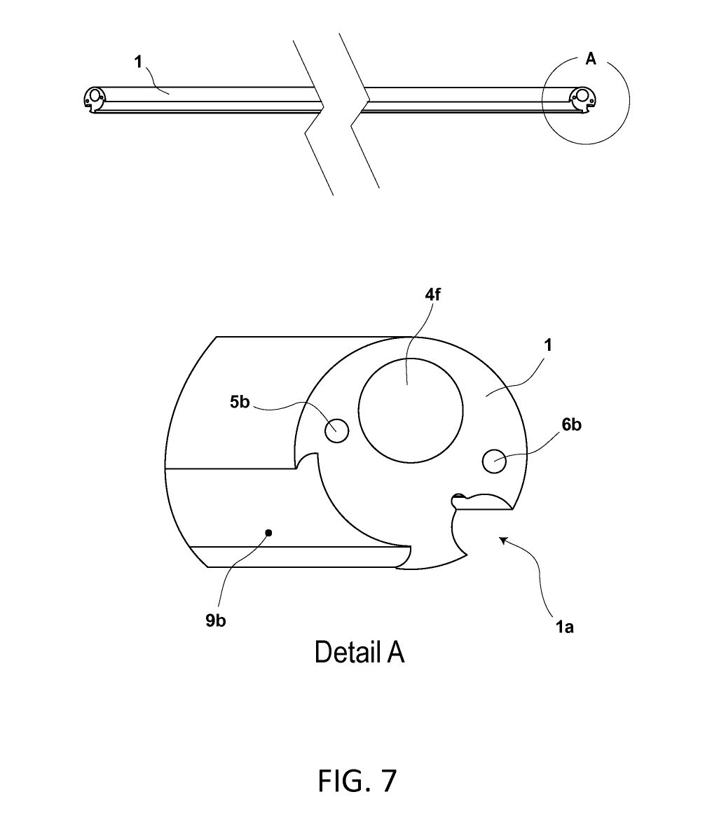

FIG. 7 illustrates a segment of an endoscope tubular body 1 and a cross-section A of the endoscope tubular body 1. The endoscope tubular body 1 of FIG. 7 is configured to receive a preformed stiffening member (not shown). The endoscope tubular body 1 has a flexible core, which may act as the main body of an endoscope (e.g., endoscope 100A-D). The flexible core 1 includes a primary axial thru port 4f extending the full length of the core 1 and at least one secondary port 5b (e.g., steering cable lumen) extending the same length. FIG. 7 illustrates another optional secondary port 6b. The outer surface of the core 1 has a generally smooth profile with the exception of the semi-cylindrical cutout section retention channel 1a, which extends substantially along the full length of the core 1. The endoscope tubular body 1 includes a curved profile stiffening member channel 9b.

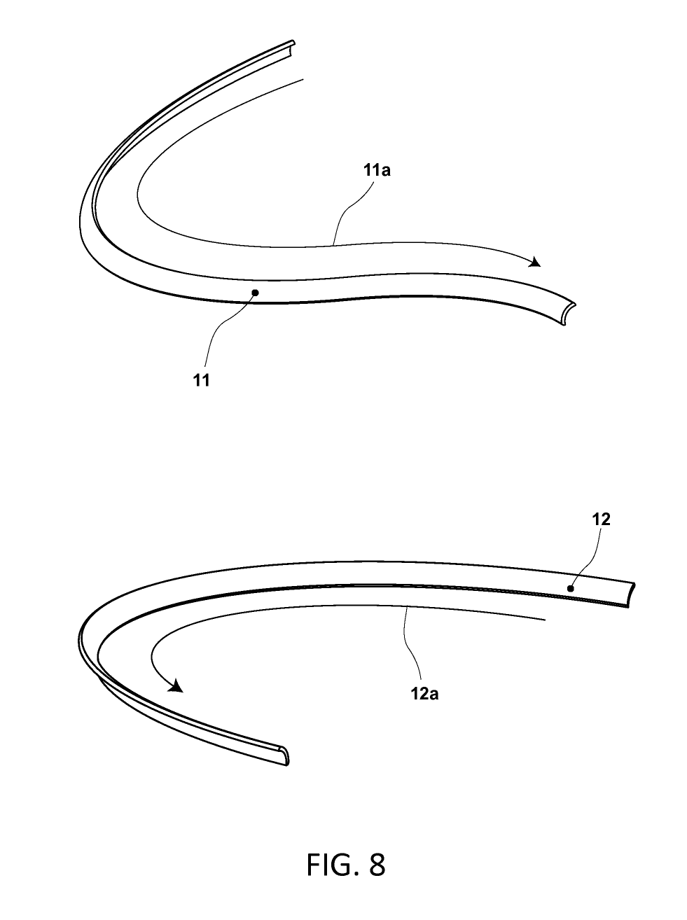

FIG. 8 illustrates two additional embodiments of preformed stiffening members 11, 12. The preformed member 11 is a shaped stiffener having multiple shaped sections and a shaped profile 11a. The preformed member 12 is a different shaped stiffener having multiple shaped sections and a shaped profile 12a. The shapes and profiles of the preformed members 11, 12 of FIG. 8 illustrate non-limiting shape embodiments of a nearly unlimited number of profiles the preformed members could have.

The optional attachment embodiments of FIGS. 5-8 can be installed within or as a structural feature on the outer surface of the endoscope tubular body. The optional attachment can be held in place mechanically with a removable attachment mechanism, glued in place, held in place with friction, magnetized, or attached in some other way. The optional attachment may be permanently affixed to the endoscope tubular body or removably affixed, for example, by using mechanisms described with respect to FIGS. 1-4.

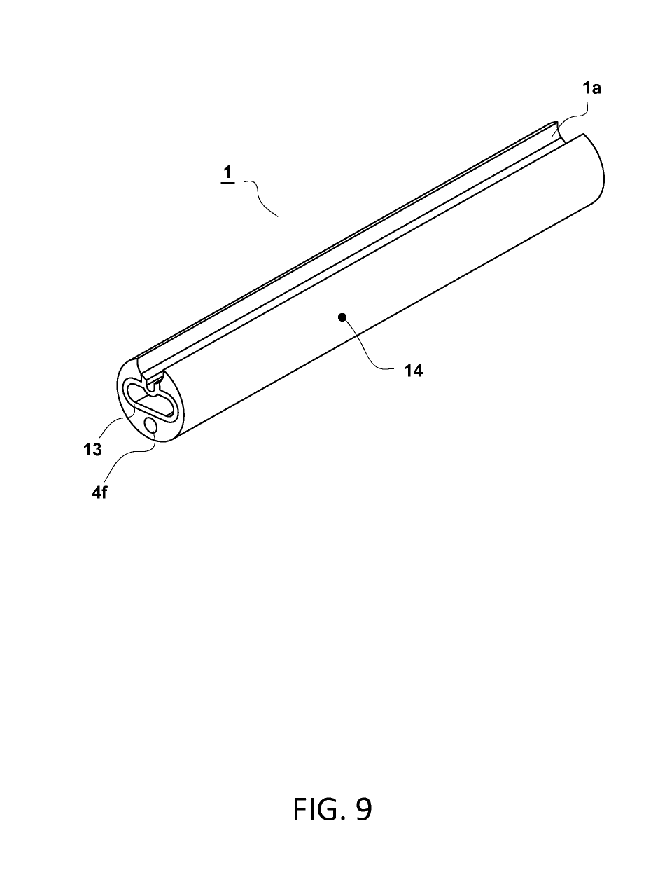

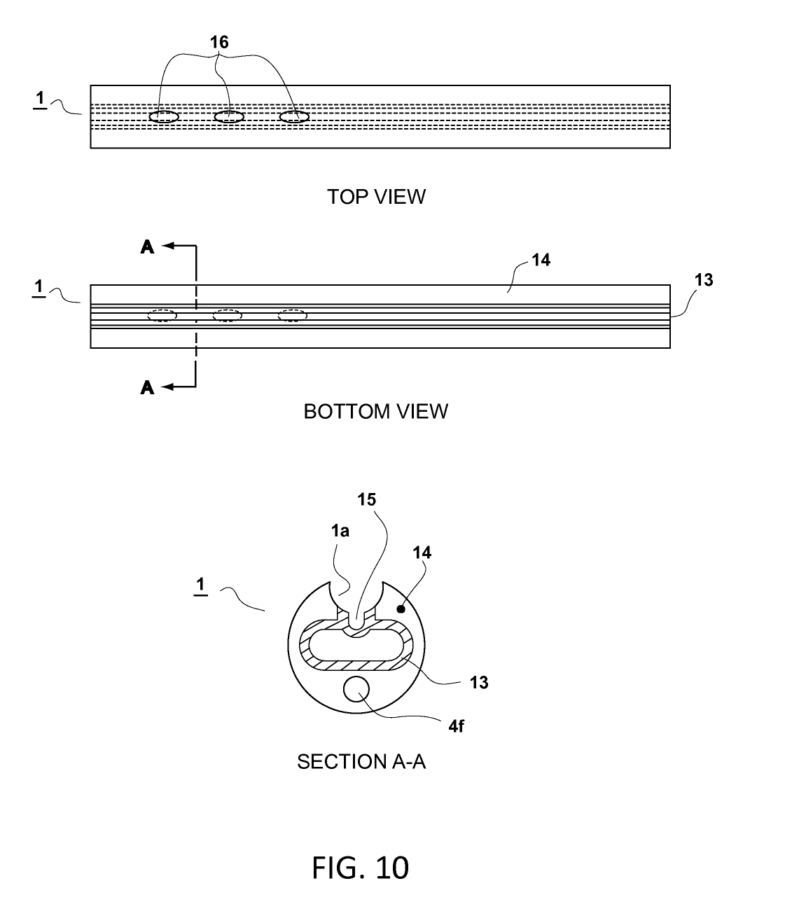

FIGS. 9 and 10 illustrate one more embodiment of an endoscope tubular body 1. FIG. 9 is a perspective view of the endoscope tubular body segment 1. The tube body segment 1 has a rigid or semi-rigid inner portion 13 and a soft outer portion 14. FIG. 10 illustrates top, bottom, and cross-section views of the endoscope tubular body segment 1 of FIG. 9. The endoscope tubular body segment 1 of FIGS. 9 and 10 may optionally be formed as a disposable endoscopic instrument used for medical procedures within a human patient's body or the body of some other patient. Alternatively, the endoscope tubular body segment 1 can include some or all components formed in a non-disposable way.

In some cases, the rigid or semi-rigid inner portion 13 of the endoscope tube body segment 1 of FIGS. 9 and 10 is formed with a same material as the soft outer portion 14. In other cases, the inner and outer portions are formed with different materials. Optionally, the inner portion 13 can even be formed as a soft portion while the outer portion 14 is formed as a rigid or semi-rigid portion.

In some cases, the inner and outer portions may be differently colored. The different colors may provide sufficient contrast to help orient the tube segment for a medical practitioner or the different colors may provide other information. For example, the different colors may indicate to a medical practitioner that a retention channel has an inserted auxiliary tool or that such a tool is absent. The different colors may be further used to indicate tool diameter, material, insertion depth, or other characteristics.

In certain instances and in some procedures, the endoscopic tubular body 1 will exhibit some predetermined degree of stiffness. Alternatively, or in addition, the tool body will exhibit some predetermined degree of torque stability. The mechanical nature of the endoscopic tubular body segment 1 of FIGS. 9 and 10 is configured such that mechanical control motions applied by a medical practitioner are transmitted down the some portion or the entire length of the endoscope tubular body. In other words, as the medical practitioner rotates a control mechanism (e.g., a control handle) of the instrument, the tubular body will exhibit substantially the same rotation.

The torque stable nature of an endoscope permits a medical practitioner to position the instrument at a desired location within the body of a patient, even as the endoscopic tubular body may twist and turn along its length. During or after the insertion, the medical practitioner will remain able to adjust the instrument's orientation with substantial certainty for diagnostic or therapeutic purposes.

In conventional endoscopes, a desired degree of torque stability and mechanical controllability is obtained with a tubular body that employs a multi-clad armor sheath having interlocking segments arranged in a helical-anti-helical fashion. In this arrangement, as one segment begins to twist, an opposing segment locks against it, thereby opposing the twist. The conventional arrangement is mechanically effective, but the manufacture of such a device is complex, expensive, and generally not well suited for a disposable instrument.

In an endoscope having the tubular body segment 1 of FIGS. 9 and 10, several problems of a conventional endoscope tubular body are solved. The tubular body segment 1 of FIGS. 9 and 10 may be disposable. The tubular body segment 1 may be manufactured more easily and at a lower cost. Furthermore, the tubular body segment 1 of FIGS. 9 and 10 may include other endoscopic attachment features as described herein.

In the endoscope tubular body segment 1 of FIGS. 9 and 10, a tubing segment is formed as a portion of a disposable endoscope. The tubular body segment 1 includes a semi-flexible tube having one or more lumens extending the full length or alternatively, a portion of the length. The tubular body segment 1 outer portion includes features for retention of an external tube and wire release mechanism (e.g., FIGS. 1-4).

In some embodiments, the tubular body segment 1 is formed as a coextruded body. The body parts, as described herein, may include two (or more) different or same materials. The body parts may be molded in different shapes and joined (e.g., fused, interlocked, pressure fit, shape fit, etc.) together to form the tubular body segment 1. The tubular body segment 1 can be formed in a single manufacturing process. Alternatively, the inner part 13 and outer part 14 can be formed separately and the tubular body segment 1 can be later assembled.

In one embodiment of a tubular body segment 1 of the type illustrated in FIGS. 9 and 10, the outer portion 14 is formed from a soft material. For example, the outer portion is formed using a medical grade silicone rubber or other material having similar properties. The outer portion 14 is soft and semi-flexible. The outer portion 14 may also have a low surface energy, which may limit the adhesion of body fluids. Additionally, the soft outer portion 14 may also facilitate attachment of an external tool (e.g., a feeding tube, catheter, etc.) to the outer surface of the endoscope tubular body segment 1.

Also in the example of the tubular body segment 1 having a soft outer portion 14, the inner portion 13 is a rigid or semi-rigid portion. The inner portion 13 is formed from material that is stiffer than the material used to form the outer portion 14. For example, the inner portion 13 may be formed from nylon, glass filled nylon (i.e., for increased stiffness), or from some other material. The inner portion 13 may be formed with a predetermined shape or the inner portion 13 may be formed from a semi-rigid, flexible, shape-holding material (e.g., medical grade rubber, silicone, or polyethylene encapsulated metal).

Further in the example of the tubular body segment 1 having a rigid or semi-rigid inner portion 13 and soft outer portion 14, the geometric profile of the inner portion 13 may be formed to have a predetermined, desired effect on the mechanical performance of the endoscope. For example, if a procedure calls for a long tubular body, the profile of the rigid or semi-rigid inner portion 13 may be different from a tube used in procedures calling for a short tubular body.

In some embodiments, the inner portion 13 may be formed with a feature 15 that cooperatively facilitates a mechanism to dislocate an external tool from the tubular body segment 1. Similar features are described herein (e.g., the retention channel 1a and cable assembly 3 of FIGS. 4A-4D). The feature 15 may benefit from a reduced friction between the dislocation mechanism and the inner portion 13 material surface. Such a reduced friction can help to reduce the chance of disrupting the instrument's global location during separation of a tool from the outer portion 14. A low friction material cooperatively combined with a certain rigidity of the inner portion 13 may be similarly beneficial in an assembly process.

In some embodiments of the example, such as the embodiment illustrated in FIGS. 9 and 10, the inner portion 13 is formed to include a central lumen 4f throughout its length. In other embodiments, the lumen 4f is not in the center of the inner portion 13. In still other embodiments, a plurality of lumens or no lumens are formed in the inner portion 13. A lumen in the inner portion 13 can be used to pass wires for electrical interface, steering cables, and other tools or devices through some part of all of the lumen.

Embodiments of the tubular body segment 1 of FIGS. 9 and 10 may also include desired apertures 16. In FIG. 10, the apertures 16 are illustrated as formed in the inner portion 13. Apertures 16 may also be formed in the outer portion 14. The apertures 16 may be used to align the inner and outer portions, pass light, pass tools, or the apertures 16 may be used for some other purpose.

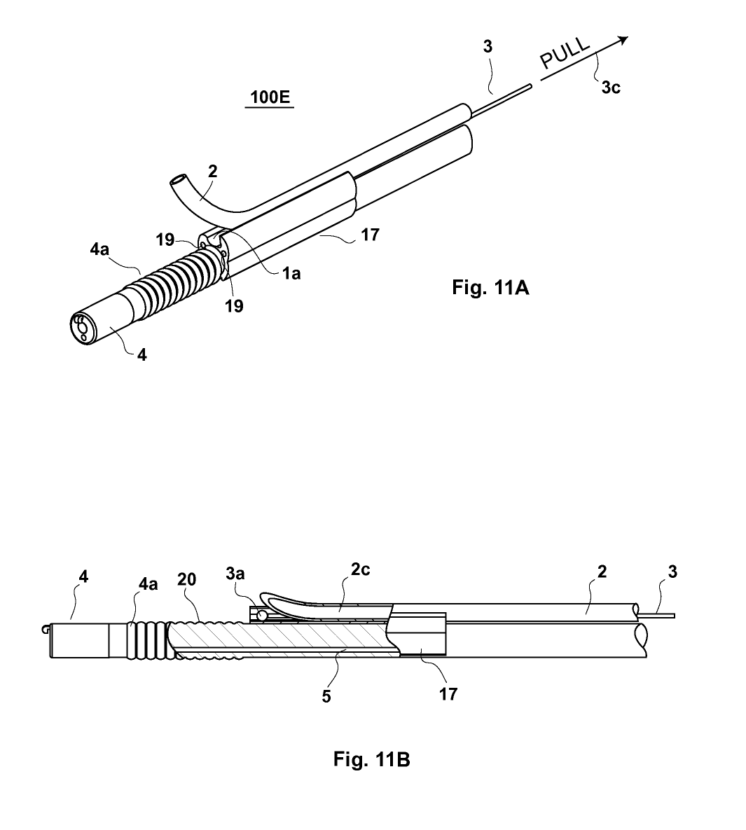

FIG. 11A is a perspective view of a distal end of a steerable endoscopic device 100E having attached thereto a medical device delivery tool 17. The steerable endoscopic device 100E includes a flexible end assembly 4 having flexible baffles 4a. Below the flexible baffles 4a, steering mechanism permits the end assembly to move in one or more directions.

The medical device delivery tool 17 is attached to the tubular body of the endoscopic device 100E. In the embodiment of FIG. 11A, the medical device delivery tool 17 has a semi-cylindrical structure. The medical device delivery tool 17 and the tubular body of the endoscopic device 100E are pressure fit together in a cooperative arrangement.

The medical device delivery tool 17 also includes a retention channel 1a. In the embodiment of FIG. 11A, an auxiliary tool 2 is carried in the retention channel 1a. A cable assembly 3 is positioned in the retention channel 1a below the auxiliary tool 2. In some embodiments, the cable assembly 3 includes a shaped end fixture (not shown). In other embodiments, the distal end of the cable assembly 3 may be temporarily attached to both the retention channel 1a and the auxiliary tool 2. The attachment for example may be via an adhesive, a temporary weld, or by some other mechanism. In this manner, the auxiliary tool is temporarily bound to the medical device delivery tool 17.

In a medical procedure, the distal end of the steerable endoscopic device 100E is advanced into the body of a patient. Concurrently, the medical device delivery tool 17 also enters the body of the patient. The medical practitioner steers the endoscopic device 100E using visualization components of the endoscopic device 100E. When the medical practitioner determines that a suitable location has been reached, the medical practitioner pulls a cable assembly 3 in direction 3c. As the cable assembly 3 is pulled, the auxiliary tool 2 is released from the medical device delivery tool 17.

In some cases, the release of the auxiliary tool 2 is carried out when a shaped end fixture 3a (not shown) is pulled through the retention channel 1a below the auxiliary tool 2 and withdrawn from the body of the patient. In such cases, the auxiliary tool 2 may have been compression fit into the retention channel 1a and held by friction. The shaped end fixture 3a passing below the auxiliary tool 2 overcomes the friction of the compression fit and displaces the auxiliary tool 2 from the channel 1a.

In other cases, the release of the auxiliary tool 2 is carried out when the temporary bond between the cable assembly 3, the retention channel 1a, and the auxiliary tool 2 is broken. In this case, the auxiliary tool 2 may have a smaller diameter than the retention channel 1a, and merely breaking the bond is sufficient to release the tool 2 from the medical device delivery tool 17. Alternatively, the auxiliary tool 2 may not even have a tubular shape. Instead, the distal end of the tool 2 may have some other shape or size that is only engaged in the retention channel 1a at the distal end. When the bond is broken, the tool 2 is released from the medical device delivery tool 17.

The medical device delivery tool 17 illustrated in FIG. 11A may optionally include one or more of lumens 19. The lumens 19 may be contain steering cables, lighting means, visualization means, or some other tool. The lumens may be used to pass air, water, or some other gas other fluid in one or two directions. The lumens may be used for other purposes as well.

FIG. 11B is a side view, section view of the medical device delivery tool apparatus embodiment of FIG. 11A. The distal end of the steerable endoscopic device 100E having attached thereto a medical device delivery tool 17 is shown. From the flexible baffles 4a, a sheath 20 is exposed. The sheath 20 may house a flexible spring mechanism, a plurality of interlocking segments arranged in a helical-anti-helical fashion, or some other flexible means configured as part of the steering mechanism of the distal end of the endoscopic device 100E.

The section view of FIG. 11B illustrates a steering cable 5 inside the tubular body of the endoscopic device 100E.

The section view of FIG. 11B illustrates an inside cavity 2 of the auxiliary tool 2. In some cases, the auxiliary tool 2 has a closed distal end (FIG. 11B), and in other cases, the auxiliary tool 2 has an open distal end (FIG. 11A). In still other cases, the distal end of the auxiliary tool 2 may have a different structure.

The section view of FIG. 11B illustrates a spherical shaped end fixture 3a formed at the distal end of the cable assembly 3. In other embodiments, the shaped fixture 3a may have a wedge shape, a teardrop shape, or some different shape.

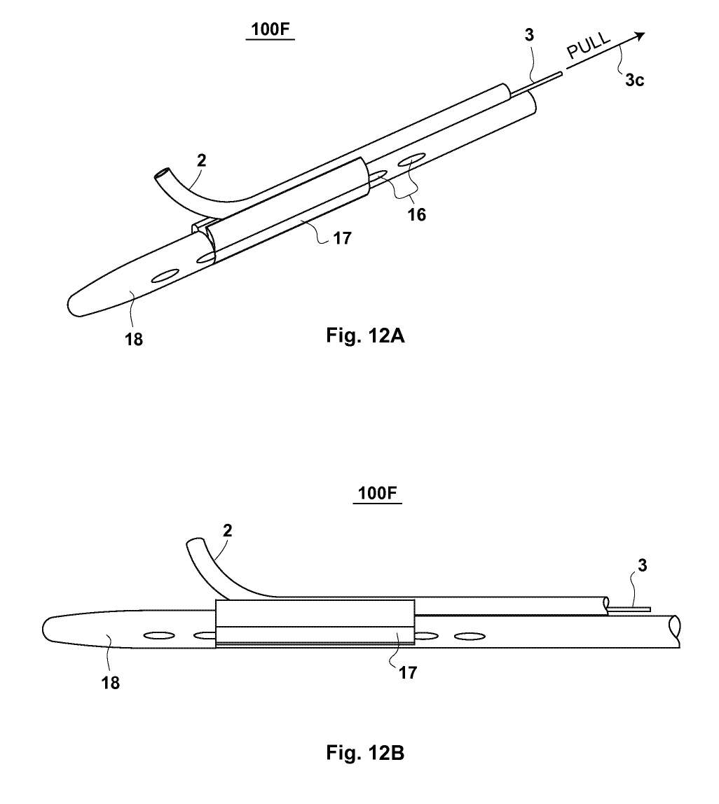

FIG. 12A is a perspective view of a distal end of a medical device placement tool arrangement 100F. FIG. 12B is a side view of the medical device placement tool arrangement embodiment 100F of FIG. 12A. The arrangement 100F includes a medical device placement tool 18 having attached thereto a medical device delivery tool 17. In some cases, the medical device delivery tool is less than 10 centimeters long.

The medical device placement tool 18 may be formed as a flexible rod, a rigid rod, an elongated shank, a tube, a cylinder, or in some other shape. As illustrated in FIG. 12A, the medical device delivery tube 17 is coupled longitudinally to the medical device placement tool 18. Other arrangements are possible.

The medical device placement tool 18 of FIG. 12A is illustrated having apertures 16, but such apertures are optional. For example, the medical device placement tool 18 may have a solid structure, a hollow structure, or a structure formed from a plurality of materials. In some cases, the medical device placement tool 18 passes a lubricant or therapeutic agent (e.g., an antibiotic, a disinfectant, a medicine, etc.) through the apertures 16. In other cases, the medical device placement tool 18 is coupled to a suction device, and material is drawn through the apertures 16. In some cases, the medical device placement tool 18 is used to place a flexible drainage tube medical device in the body of the patient.

The embodiment of FIG. 12A includes a cable assembly 3. When the cable assembly 3 is pulled in direction 3c, the auxiliary tool 2 is released from the medical device delivery tool 17.

In FIGS. 12A and 12B, the auxiliary tool 2 is illustrated having a distal end separated from the medical device placement tool 18. In some embodiments, the illustration conveys teaching of the placement of the auxiliary tool 2 into a retention channel of the medical device delivery tool 17. In other embodiments, the illustration conveys teaching of the separation of the auxiliary tool 2 from the medical device delivery tool 17. The auxiliary tool 2 is illustrated in FIGS. 12A and 12B as extending through a retention channel and past in an of the medical device delivery tool 17, but such placement is optional. For example, in some embodiments the end of the auxiliary tool 2 is substantially aligned with the end of the medical device delivery tool. In other embodiments, the end of the auxiliary tool 2 is placed such that the tool does not horizontally fill the entire retention channel.

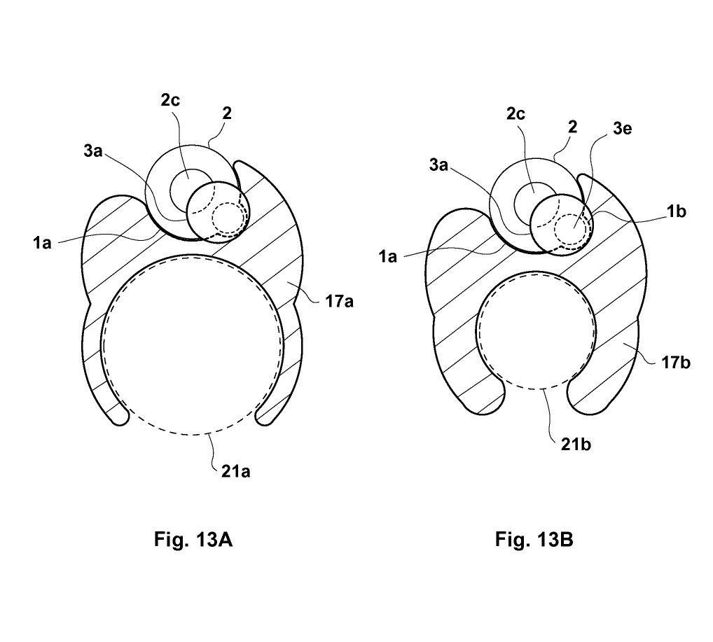

FIGS. 13A and 13B are, respectively, cross-sectional views of two different medical device delivery tool embodiments 17a, 17b. The cross-sectional views of the medical device delivery tool embodiments 17a, 17b illustrate an auxiliary tool 2 placed in a retention channel 1a. The retention channel is axially formed as a semi-cylindrical conduit in the body of the medical device delivery tool 17a, 17b. Various figures illustrate such retention channel with different views.

As illustrated in FIGS. 13A and 13B, the retention channel 1a may also be formed having a secondary channel 1b. In such embodiments, the retention channel 1a is arranged for cooperative coupling with the auxiliary tool 2. The auxiliary tool 2 may be held in the retention channel 1a by friction. In addition, or alternatively, the auxiliary tool 2 may be formed from a flexible material that is compressed when the tool 2 forced into the retention channel 1a. In still other embodiments, the auxiliary tool 2 is loosely aligned in the retention channel 1a.

In FIGS. 13A and 13B, the auxiliary tool 2 is illustrated as having an optional cavity 2. In some embodiments, the auxiliary tool 2 is a catheter or some other tube wherein the cavity 2c passes longitudinally through the entire tool 2. The distal end of the auxiliary tool 2 may be open or closed. The distal end of the auxiliary tool 2 in FIGS. 13A and 13B is shown open.

The retention channel 1a of FIGS. 13A and 13B also include a secondary channel 1b. This secondary channel 1b is formed for placement of the cable assembly 3, which may include a separation means 3e. The separation means 3e may include any type of structure arranged to separate an auxiliary tool 2 from its position in a retention channel 1a of a medical device delivery tool. For example, one separation structure is a shaped end fixture 3a (e.g., FIG. 4C). Another separation structure is a bond that temporarily couples the auxiliary tool 2 to the medical device delivery tool 17, 17a, 17b. Other types of separation structures are also possible.

In embodiments where the cable assembly includes a shaped end fixture, the shaped end fixture separates the auxiliary tool 2 from the medical device delivery tool 17, 17a, 17b when the fixture passes below the auxiliary tool 2 as the cable assembly 3 is pulled. In embodiments where the cable assembly includes a bond, the auxiliary tool 2 is separated from the medical device delivery tool 17, 17a, 17b when the cable assembly 3 is pulled with a force sufficient to break the bond.

In FIG. 13A, the medical device delivery tool 17a has a coupling mechanism with a selected diameter 21a. In some cases, the selected diameter 21a is less than 20 millimeters. In FIG. 13B, the medical device delivery tool 17b has a coupling mechanism with a different selected diameter 21b. In some cases, the selected diameter 21b is less than 10 millimeters. FIGS. 13A and 13B illustrate one mechanism wherein the delivery tool can be coupled to a particular medical device placement tool. The medical device placement tool can be a steerable endoscope (100A-100E), a medical device placement tool arrangement (100F), or some other structure. In some cases the medical device delivery tool 17, 17a, 17b is permanently coupled to the medical device placement tool, and in other cases the coupling is temporary.

As described herein, the terms “rigid” and “semi-rigid” may be interchanged. Accordingly, “rigid” device is not necessarily completely unbendable. Instead, a rigid device or a rigid portion of a device has a desired degree of stiffness. That is, a device that is “rigid” or “semi-rigid” is a device that resists deformation to a desired degree. The desired degree of rigidity may be measured, for example, in units such as foot pounds per inch or some other units. One device may be more rigid than another device. The increased (or decreased) rigidity may be caused by the devices being formed from different materials, from materials having different physical or chemical properties, or for some other reason.

The embodiments described herein may be assembled in new devices or they may be useful as accessories to existing endoscopes. In the case of a conventional endoscope, one primary component 1 having the features described herein could be installed over the tube body of the conventional endoscope. Auxiliary tubes could be attached to the core (i.e., affixed to the endoscope tubular body), and directed to a site in the body of a patient for tube placement. Removal of the medical tube (e.g., catheter) from the core could be performed using the methods described herein, while the primary component 1 remains attached to the endoscope body. It is to be understood that the invention is not limited to the particular embodiments described, which may of course vary. It is also to be understood that the terminology used herein is for the purpose of describing particular embodiments only, and is not intended to be limiting, since the scope of the present invention is limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

As used herein and in the appended claims, the singular forms “a”, “an”, and “the” include plural referents unless the context clearly dictates otherwise. It should also be noted that the term “or” is generally employed in its sense including “and/or” unless the content clearly dictates otherwise.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The headings and Abstract of the Disclosure provided herein are for convenience only and do not interpret the scope or meaning of the embodiments.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

1. An endoscopic device, comprising:
a tubular body having a retention channel substantially extending axially along the tubular body;
a medical device removably arranged in the retention channel and held in place by friction; and
a separation mechanism arranged between the medical tube and a surface of the retention channel.

2. The endoscopic device of claim 1 wherein the separation mechanism includes a cable assembly and a shaped end fixture.

3. The endoscopic device of claim 2 wherein the shaped end fixture has a ball shape, a teardrop shape, or a wedge shape.

4. The endoscopic device of claim 1 wherein the separation mechanism includes a cable assembly and a temporary bond between the retention channel and the medical device.

5. The endoscopic device of claim 1 wherein the tubular body has a plurality of retention channels.

6. The endoscopic device of claim 1 wherein the tubular body is a separate component from an endoscope, and the tubular body and the endoscope are assembled together as an integrated medical device delivery tool.

7. A method to place a medical tube in a patient's body with an endoscope, the endoscope having arranged therewith a tubular body structure, the tubular body structure having an integrated retention channel substantially extending axially along the tubular body structure, a medical tube removably fit in the retention channel and held in place by friction, and a separation mechanism arranged between the medical tube and a surface of the retention channel, the separation mechanism including a cable assembly, the method comprising:
positioning a distal end of the endoscope in a patient's body;
drawing the cable assembly in a direction away from the patient, the drawing performed to release the medical tube from the retention channel; and
removing the endoscope from the patient.

8. The method of claim 7, comprising:
passing a liquid through the medical tube.

9. The method of claim 7, comprising:
steering a distal end of the endoscope by manipulating at least one linkage at a proximal end of the endoscope.

10. The method of claim 7, comprising:
drawing a second cable assembly in a direction away from the patient, the drawing performed to pull a shaped end fixture between a second medical tube and a surface of a second retention channel thereby releasing the second medical tube from the second retention channel.

11. A medical device delivery tool, comprising:
a substantially tubular body having a retention channel formed therein, the retention channel extending axially along the tubular body, the retention channel arranged to receive a medical device;
a medical device removably arranged in the retention channel and held in place by friction; and
a separation mechanism arranged between the medical device and a surface of the retention channel.

12. The medical device delivery tool of claim 11 wherein the substantially tubular body has a secondary channel formed within the retention channel, the secondary channel extending axially along the tubular body, the secondary channel arranged to receive a cable assembly of the separation mechanism.

13. The medical device delivery tool of claim 12 wherein the cable assembly includes a shaped end fixture, the shaped end fixture arranged to be drawn between the medical device and the retention channel.

14. The medical device delivery tool of claim 12 wherein the cable assembly includes a stainless steel cable.

15. The medical device delivery tool of claim 11 wherein the substantially tubular body has a plurality of retention channels.

16. The medical device delivery tool of claim 11, comprising:
a semi-cylindrical coupling mechanism having a selected diameter, the semi-cylindrical coupling mechanism arranged to be removably attached to a substantially tubular body.

17. The medical device delivery tool of claim 16 wherein the selected diameter is between 10 millimeters and 20 millimeters.

18. The medical device delivery tool of claim 16 wherein the substantially tubular body is a body of an endoscope.

19. The medical device delivery tool of claim 16 wherein the substantially tubular body is a solid flexible rod.

20. The medical device delivery tool of claim 16 wherein the substantially tubular body is less than 10 centimeters long.
